(19) United States
(12) Patent Application Publication
(10) Pub. No.: US 2008/0260807 A1
(43) Pub. Date: Oct. 23, 2008

(54) Water-Soluble Films Comprising Nicotine

(75) Inventors: Andrew John Sharp, Norfolk (GB); Edward Zbygniew Nowak, Cambridge (GB)

Correspondence Address: MCDERMOTT WILL & EMERY LLP, 18191 VON KARMAN AVE., SUITE 500, IRVINE, CA, 92612-7108, US

(73) Assignee: Bio Progress Technology Limited, Cambridge (GB)

(21) Appl. No.: 11/912,658

(22) PCT Filed: Apr. 25, 2006
(86) PCT No.: PCT/GB06/01503

§ 371 Date: Apr. 2, 2008

(30) Foreign Application Priority Data
Apr. 25, 2005 (GB) 0508306.8

(51) Int. Cl. A61K 9/70; A61K 31/4439; A24B 3/14; A61P 25/34

(52) U.S. Cl. 424/443; 514/343; 131/369

(57) ABSTRACT

A water-soluble film comprising nicotine, wherein said nicotine may be any pharmacologically effective form of nicotine present at one or more locations of the film defined by the application of a nicotine-containing fluid to at least one surface of a preformed water-soluble film.

The disclosed invention relates to water-soluble films comprising nicotine, methods of making the same, dosage forms comprising such films, and uses of such films.

Nicotine addiction is a world-wide problem, arising primarily via smoking tobacco based products, and to a lesser extent via the use of other forms of tobacco, such as chewing tobacco. This results in a range of related health problems and diseases, and creates an associated financial burden generated by the provision of health care and treatment for these health problems.

There have therefore been considerable efforts made to reduce the prevalence of smoking. A number of products have been developed to assist those addicted to nicotine in weaning themselves off their addition. Such products include chewing gums, which administer amounts of nicotine orally, and patches, which are affixed to the skin and administer nicotine topically. However, these products are of limited effectiveness, due to the time taken for the nicotine to enter the user's system and have the required pharmacological effect. The delay between the utilisation of the product and the pharmacological effect of the administered nicotine provides a period after utilisation of the product in which the addict's craving for nicotine has not yet been sated. During this period, the addict is more likely to sate the addiction through the original means giving rise to the addiction, e.g. by smoking a cigarette, thereby negating or mitigating any benefits of the product. The problem is particularly prevalent in those individuals whose addiction derives form smoking products containing nicotine, as smoking results in nicotine being delivered via inhalation, such that the nicotine quickly enters the blood stream and thereby provides a rapid pharmacological effect; smokers are used to, and expect, a rapid pharmacological effect from a nicotine based product.

Further, such a delay may be particularly problematic for products such as chewing gums, for which it is intended a single unit of a product be used in place of a single cigarette (or other form of nicotine). This is because an addict is likely to use the product only when prompted by a craving for nicotine. Therefore, the cravings which occur during the period between administration of the product and the pharmacological effect of the nicotine are likely to be particularly acute, such that abstention from the original form of nicotine is even less likely.

There are also individuals who use such products with no intention of ending their addiction, but who instead want relief from the cravings of the addiction for a period of time, for example if the individual is required to spend a period of time in an environment in which the regular form of their habit is not permitted, e.g. during an aeroplane flight on which smoking is not permitted. The same problem of delayed effect arises in this instance, as there will be a period during which the user does not obtain the desired relief.

A further problem exists in that the forms of nicotine which are used in currently known products are expensive. Additionally, certain forms of nicotine, such as nicotine oil are volatile and difficult to handle, which, in addition to problems arising from the inherent dangers of using such materials, further increases the cost of producing the currently known products. This cost can discourage addicts from using such products. Hence, some known methods rely on using large amounts of more volatile forms of nicotine.

Single unit dosage forms made from water-soluble films comprising an active ingredient are known. However, such films have not previously been considered suitable for administering pharmacological substances due to a number of processing difficulties. In particular, the aggregation or conglomeration of some active ingredients means that there may be an uncontrollably, unknowably uneven distribution of active ingredient throughout the film, resulting in some parts of the film containing no active ingredient, whilst other parts of the film may contain a high density of active ingredient. This may be overcome to an extent by providing very high concentrations of active material, but this results in an increased cost, and may be unsuitable when working with pharmacologically active particles, for which a precise dose, neither too high or too low, is required.

Further, the known techniques for casting films require relatively long drying times, which further promotes aggregation of the active ingredient, and may result in the exposure of the active ingredient to atmospheric, or even increased, moisture levels and temperature, which may degrade the active ingredient. This is particularly problematic for certain forms of nicotine, such as nicotine bitartrate dihydrate (hereinafter “NBD”), which are subject to crystallisation. Crystallisation of the active material further increases the lack of controlled distribution of the active material, and can lead to the active material precipitating out of the water-soluble film altogether. In some cases, the crystallisation of nicotine will create a residue of crystallised nicotine on the surface of the film, which will then abrade from the surface.

The use of water-soluble films has further been considered especially unsuitable for the provision of nicotine dosage forms, given that certain polymers used in the formation of such films actively promote the crystallisation of some forms of nicotine, particularly the forms of nicotine most favourable for use in products for assisting nicotine addicts in weaning themselves from their addiction, such as NBD. Also, certain forms of nicotine will crystallise even after the formation of such films, due to handling and prolonged exposure to the atmosphere of such films.

The provision of dosage forms made from water-soluble films is discussed in WO 2005/079750 (the disclosures of which are incorporated herein by reference). However, water-soluble films comprising nicotine are not contemplated in WO 2005/079750, nor are the specific problems arising in relation to nicotine addressed.

### SUMMARY OF INVENTION

An embodiment of the present invention provides a water-soluble film comprising nicotine, wherein said nicotine may be any pharmacologically effective form of nicotine present at one or more locations of the film defined by the application of a nicotine-containing fluid to at least one surface of a preformed water-soluble film.

A further aspect of the present embodiment optionally provides that said film is ingestible.

A further aspect of the present invention optionally provides that said pharmacologically active form of nicotine comprises one or more of: a nicotine oil, a nicotine salt or a complex of nicotine. Optionally said pharmacologically active form of nicotine is nicotine bitartrate dihydrate. Optionally, said nicotine salt is a: hydrochloride; dihydrochloride; sulphate; tartrate; ditartrate; zinc chloride; salicylate; alginate; ascorbate; benzoate; citrate; edetate; fumarate; lactate; maleate; oleate; or sorbate. Optionally, said pharmacologically active form of nicotine comprises a microencapsulated form of nicotine.

A further aspect of the present embodiment optionally provides that said water-soluble film comprises a cellulose ether.

A further aspect of the present embodiment optionally provides that said water-soluble film comprises a polymer based film comprising one or more of: hydroxy propylmethyl cellulose; hydroxy propyl cellulose; hydroxy ethyl methyl cellulose; hydroxyethyl cellulose; methyl cellulose; carboxymethyl cellulose; sodium carboxymethyl cellulose; a salt or derivative of any of the aforesaid cellulose ethers; cellulose acetate phthalate; hydroxypropyl methyl cellulose phthalate; hydroxypropyl methyl cellulose acetate succinate; ethyl cellulose; carboxymethyl hydroxyethyl cellulose; and a sodium salt of a carboxymethyl hydroxyethyl cellulose.

A further aspect of the present embodiment optionally provides that said film further comprises a plasticiser. Optionally said plasticiser comprises one or more of: polyols; glycols; acetins; carboxylic acid; and esters of carboxylic acid. More particularly, optionally said plasticiser comprises one or more of: polyethylene glycol; glycerine, triacetin; citric acid; and triethyl citrate. Optionally said plasticiser or plasticisers are present in an amount up to 40% of the solid film. More particularly, optionally said plasticiser or plasticisers are present in an amount up to 20% of the solid film.

A further aspect of the present embodiment optionally provides that said nicotine is evenly distributed through the said film.

A alternative aspect of the present embodiment optionally provides that said nicotine is present in a concentration gradient through said film. Optionally, the greatest concentration of nicotine is located a surface of a film to which the nicotine-containing fluid is applied, and said concentration gradient provides that the concentration of nicotine decreases with distance away from said surface.

A further aspect of the present embodiment optionally provides that said nicotine is present in one or more locations arising from application of said fluid on at least one discrete area of a surface of said film such that said film surface comprises areas of demarcation, wherein one or more of said areas comprises a measurably lower concentration of said nicotine in relation to one or more other said areas. Optionally, said one or more areas comprises a concentration of said nicotine of 0%.

A further aspect of the present embodiment optionally provides that said film further comprises one or more additional active materials. Optionally, at least one of said one or more additional active materials has a different distribution on a surface of said film in comparison with the distribution of said nicotine. Optionally, said one or more additional active materials is present at locations arising from the application of a fluid comprising said one or more additional active materials, such that said film surface comprises areas of demarcation, wherein one or more of said areas comprises a measurably lower concentration of said one or more additional active materials in relation to one or more other said areas. Further, said fluid comprising nicotine and said fluid comprising one or more additional active materials may be different fluids. Optionally, said one or more areas comprises a concentration of said one or more additional active materials of 0%. Optionally said areas of demarcation comprising one or more additional active materials exactly or substantially match the locations at which nicotine is present. Alternatively, said areas of demarcation comprising one or more additional active materials overlap with the locations at which nicotine is present. Still alternatively, said areas of demarcation comprising one or more additional active materials partially overlap with the locations at which nicotine is present. Further alternatively, said areas of demarcation comprising one or more additional active materials are distinct from the locations at which nicotine is present.

Optionally, one or more of said active materials comprises caffeine. Optionally, said one or more additional active materials comprises an analgesic. Optionally, said one or more additional active materials comprises a breath freshening agent. Preferably, said breath freshening agent comprises menthol. Optionally, said one or more additional active materials comprises an oral cleansing agent. Preferably, said oral cleansing agent comprises one or more of a quaternery or ammonium base.

A further aspect of the present embodiment optionally provides that said film further comprises one or more of: a colourant; an emulsifier; an humectant; a defoamer; and an anti-block agent.

An alternative embodiment of the present invention provides a dosage form comprising a film in accordance with any of the above described embodiments.

A further aspect of the present embodiment optionally provides that said dosage form comprises a single layer of said film.

A further aspect of the present embodiment optionally provides that said dosage form comprises an enrobed tablet, wherein said tablet is enrobed by a film in accordance with any of claims 1-30.

A alternative aspect of the present embodiment optionally provides that dosage form comprises a pharmaceutical capsule, wherein the walls of said capsule comprise a film in accordance with any of claims 1-34.

A alternative aspect of the present embodiment optionally provides that said dosage form comprises multiple layers of film, wherein one or more of said layers comprises a film in accordance with any of claims 1-30. Optionally, two or more layers of said film comprise nicotine. Further, optionally, at least one of said two or more layers comprises nicotine present at one or more locations arising from application of said fluid on at least one discrete area of a surface of said layer such that said layer surface comprises areas of demarcation, wherein one or more of said areas comprises said nicotine; and wherein said one or more areas of demarcation comprising nicotine exactly match, in 2-dimensional space, the one or more locations on said second layer at which said nicotine is present. Alternatively, optionally, at least one of said two or more layers comprises nicotine present at one or more locations arising from application of said fluid on at least one discrete area of a surface of said layer such that said layer surface comprises areas of demarcation, wherein one or more of said areas comprises said nicotine; and wherein said one or more areas of demarcation comprising nicotine overlap with one or more locations on said second layer at which said nicotine is present. Still alternatively, optionally, at least one of said two or more layers comprises nicotine present at one or more locations arising from application of said fluid on at least one discrete area of a surface of said layer such that said layer surface comprises areas of demarcation, wherein one or more of said areas comprises said nicotine; and wherein said one or more areas of demarcation comprising nicotine partially overlap with the one or more locations on said second layer at which said nicotine is present. Still further alternatively, optionally, at least one of said two or more layers comprises nicotine present at one or more locations arising from application of said fluid on at least one discrete area of a surface of said layer such that said layer surface comprises areas of demarcation, wherein one or more of said areas comprises said nicotine; and wherein said one or more areas of demarcation comprising nicotine are wholly offset from the one or more corresponding locations on said second layer at which said nicotine is present.

Optionally, said dosage form comprises at least one layer comprising one or more additional active materials. Optionally, said at least one layer comprising one or more additional active materials is a layer different from said one or more layers comprising a film in accordance with any of the above described embodiments. Optionally, said one or more additional active materials is present at locations arising from the application of a fluid comprising said one or more additional active materials, such that said layer surface comprises areas of demarcation, wherein one or more of said areas comprises said one or more additional active materials, and wherein said one or more areas of demarcation comprising one or more additional active materials substantially or exactly match the locations at which nicotine is present on said surface. Alternatively, optionally said one or more additional active materials is present at locations arising from the application of a fluid comprising said one or more additional active materials, such that said layer surface comprises areas of demarcation, wherein one or more of said areas comprises said one or more additional active materials, and wherein said one or more areas of demarcation comprising one or more additional active materials overlap with the locations at which nicotine is present. Still alternatively, optionally said one or more additional active materials is present at locations arising from the application of a fluid comprising said one or more additional active materials, such that said layer surface comprises areas of demarcation, wherein one or more of said areas comprises said one or more additional active materials, and wherein said one or more areas of demarcation comprising one or more additional active materials partially overlap with the locations at which nicotine is present. Still further alternatively, optionally, said one or more additional active materials is present at locations arising from the application of a fluid comprising said one or more additional active materials, such that said layer surface comprises areas of demarcation, wherein one or more of said areas comprises said one or more additional active materials, and wherein said one or more areas of demarcation comprising one or more additional active materials are wholly offset from the locations at which nicotine is present.

Optionally, said nicotine is present in one or more locations arising from application of said nicotine-containing fluid on at least one discrete area of a surface of said layer such that said layer surface comprises areas of demarcation, wherein one or more of said areas comprises nicotine. Optionally, no area of a film comprising said nicotine is exposed at an external surface of said dosage form. Optionally, said nicotine is comprised only within internal film layers.

A further aspect of the present embodiment optionally provides that said dosage form comprises a film in accordance with any of the above described embodiments, wherein said film is folded so as to produce said dosage form comprising multiple layers of said film.

A further aspect of the present embodiment optionally provides that one or more of the position of said nicotine within said dosage form, the degree of exposure of said nicotine on the surface of said dosage form, and the density of film layers surrounding said nicotine are pre-determined so as to provide a desired rate and/or position of release of said nicotine in a user of said dosage form.

A farther aspect of the present embodiment optionally provides that said dosage form is intended to release the said nicotine and/or any additional active ingredients in the user's buccal cavity. Alternatively, said dosage form is arranged to administer the said nicotine and/or any additional active ingredients topically. Still alternatively, said dosage form is arranged to release the said nicotine and/or any additional active ingredients in a location selected from the user's: vaginal cavity; cranial cavity; abdominal cavity; otic cavity; uterine cavity; nasal cavity; sinus cavity; rectal cavity; oral cavity; and ophthalmic cavity.

A further aspect of the present embodiment optionally provides that said dosage form provides a dose of up to 2 mg of said nicotine. Preferably, said dosage form provides a dose of between 0.1 and 1.0 mg of said nicotine. More preferably, said dosage form provides a dose of about 0.4-0.7 mg of said nicotine.

An alternative embodiment of the present invention provides the use of a film of any of the above embodiments in the manufacture of a medicament.

An alternative embodiment of the present invention provides the use of a dosage form of any of the above embodiments in the manufacture of a medicament.

An alternative embodiment of the present invention provides the use of a film of any of the above embodiments in the manufacture of a medicament for the treatment of nicotine addiction. Optionally, said medicament provides relief from the symptoms of nicotine addiction.

An alternative embodiment of the present invention provides the use of a dosage form of any of the above embodiments in the manufacture of a medicament for the treatment of nicotine addiction. Optionally, said medicament provides relief from the symptoms of nicotine addiction.

An alternative embodiment of the present invention provides the use of a film of any of the above embodiments to prevent bad breath.

An alternative embodiment of the present invention provides the use of a dosage form of any of the above embodiments to prevent bad breath.

An alternative embodiment of the present invention provides the use of a film of any of the above embodiments for the treatment of nicotine addiction.

An alternative embodiment of the present invention provides the use of a dosage form of any of the above embodiments for the treatment of nicotine addiction.

An alternative embodiment of the present invention provides the use of a film of any of the above embodiments which provides relief to a user from the symptoms of nicotine addiction.

An alternative embodiment of the present invention provides the use of a dosage form of any of the above embodiments which provides relief from the symptoms of nicotine addiction.

An alternative embodiment of the present invention provides the use of a film of any of the above embodiments for the provision of nicotine to a user for the purpose of the user's pleasure or enjoyment.

An alternative embodiment of the present invention provides the use of a dosage form of any of the above embodiments for the provision of a dose of nicotine to a user for the purpose of the user's pleasure or enjoyment.

Optionally, any of the above uses comprises dissolving said film in the buccal cavity of the user. Optionally said film only partially dissolves in said buccal cavity.

An alternative embodiment of the present invention provides a method for producing a water-soluble film containing nicotine, wherein said nicotine may be any pharmacologically effective form of nicotine, comprising the step of applying a nicotine-containing fluid to the surface of a preformed water-soluble film. Optionally, wherein said pharmacologically active form of nicotine comprises one or more of: a nicotine oil; a nicotine salt; and a complex of nicotine. Optionally, said pharmacologically active form of nicotine is nicotine bitartrate dihydrate. Optionally, said nicotine salt comprises one or more of: hydrochloride; dihydrochloride; sulphate; tartrate; ditartrate; zinc chloride; salicylate; alginate; ascorbate; benzoate; citrate; edentate; fumarate; lactate; maleate; oleate; and sorbate. Optionally, said pharmacologically active form of nicotine comprises a microencapsulated form of nicotine.

A further aspect of the present embodiment optionally provides that said fluid is a liquid. Optionally, said liquid comprises a solution, a suspension or a micro-emulsion of said nicotine.

A further aspect of the present embodiment optionally provides that said fluid is an aqueous solution, said pharmacologically active form of nicotine is nicotine bitartrate dehydrate, and said solution further comprises hydroxyl ethyl cellulose, Pullulan, polyvinyl pyrollidone, and propylene glycol alginate.

A further aspect of the present embodiment optionally provides that said fluid is applied onto only one surface of said water-soluble film.

A further aspect of the present embodiment optionally provides that said fluid is applied via one or more means of one or more of a doctor's blade 4; extrusion; roller means; spraying; brush painting; and wiping. An alternative aspect of the present embodiment optionally provides that said fluid is applied via means of one or more spray jets. Optionally, at least two applications of said fluid are applied to said film. Preferably, each of said at least two applications of said fluid are applied to the same area on the surface of said water-soluble film. Preferably, the previous application of said fluid is allowed to at least partially dry prior to the next application of said fluid. More preferably, the previous application of said fluid is left for a period of at least 15 seconds to enable the drying of said fluid. Optionally, said film is heated to assist the drying of said fluid.

A further aspect of the present embodiment optionally provides that said fluid is applied in a specific, predetermined amount. Optionally, the amount of fluid applied is predetermined in order to provide said water-soluble film with an accurately metered dose of said nicotine. Preferably, said film is divided into individual dosage units, and wherein said fluid is applied in an amount such that each said individual dosage unit comprises a dose of said nicotine of up to 2 mg. More preferably, each said individual dosage unit comprises a dose of said nicotine of between 0.1 and 1.0 mg. More preferably still, each said individual dosage unit comprises a dose of said nicotine of 0.4-0.7 mg.

A further aspect of the present embodiment optionally provides that said fluid is applied in an even distribution on the surface of said film.

A further aspect of the present embodiment optionally provides that said fluid is applied only to one or more predetermined discrete areas of the surface of said water-soluble film. Optionally, said fluid is applied in an even distribution within said discrete predetermined areas.

A further aspect of the present embodiment optionally provides that said fluid acts as an adhesive to enable said water-soluble film to be bonded to a substrate. Optionally, said substrate comprises one or more additional layers of film. Preferably, one or more of said additional layers of film comprise one or more of films made in accordance with any of claims any of the above described embodiments, and/or are films according to any of the above described embodiments.

Optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film substantially or exactly match the location of the nicotine at one or more layers of said substrate. Alternatively, optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film overlap with the location of the nicotine at one or more layers of said substrate. Still alternatively, optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film partially overlap with the location of the nicotine at one or more layers of said substrate. Still further alternatively, optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film are wholly offset from the location of the nicotine at one or more layers of said substrate.

A further aspect of the present embodiment optionally provides that said water-soluble film is folded after the application of said solution, so as to create a multiple layer film. Optionally, said fluid acts to bind said film to itself. Optionally, said fluid is applied only to one or more predetermined discrete areas of the surface of said water-soluble film, such that when said film is folded to create a multiple layer film, said discrete area or areas of on the surface of the water-soluble film in one layer exactly match said discrete area or areas of on the surface of the water-soluble film in one layer at one or more other layers of said film. Alternatively, optionally, said fluid is applied only to one or more predetermined discrete areas of the surface of said water-soluble film, such that when said film is folded to create a multiple layer film, said discrete area or areas of on the surface of the water-soluble film in one layer overlap with said discrete area or areas of on the surface of the water-soluble film in one layer at one or more other layers of said film. Still alternatively, optionally, said fluid is applied only to one or more predetermined discrete areas of the surface of said water-soluble film, such that when said film is folded to create a multiple layer film, said discrete area or areas of on the surface of the water-soluble film in one layer partially overlap with said discrete area or areas of on the surface of the water-soluble film in one layer at one or more other layers of said film. Still further alternatively, optionally, said fluid is applied only to one or more predetermined discrete areas of the surface of said water-soluble film, such that when said film is folded to create a multiple layer film, said discrete area or areas of on the surface of the water-soluble film in one layer are wholly offset from said discrete area or areas of on the surface of the water-soluble film in one layer at one or more other layers of said film.

A further aspect of the present embodiment optionally provides that, after the application of said fluid to said water-soluble film, said film is cured. Optionally, said curing occurs at ambient humidity. Optionally, said curing occurs at ambient temperature. Alternatively, said curing is achieved by heating said substrate. Optionally, said substrate is heated to a temperature of 80° C.

A further aspect of the present embodiment optionally provides that said fluid comprises fugitive solvents.

A further aspect of the present embodiment optionally provides that, prior to the application of said fluid, said water-soluble film is made by casting and drying a water-soluble film solution.

A further aspect of the present embodiment optionally provides that, wherein, prior to the application of said fluid, said water-soluble film is made by coating or spraying a water-soluble film solution onto an edible product, and drying said water-soluble film solution to produce said water-soluble film. Optionally, said edible product is a tablet.

A further aspect of the present embodiment optionally provides that said film is ingestible.

A further aspect of the present embodiment optionally provides that said water-soluble film comprises a cellulose ether.

A further aspect of the present embodiment optionally provides that said water-soluble film comprises a polymer based film comprising one or more of: hydroxy propylmethyl cellulose; hydroxy propyl cellulose; hydroxy ethyl methyl cellulose; hydroxyethyl cellulose; methyl cellulose; carboxymethyl cellulose; sodium carboxymethyl cellulose; a salt or derivative of any of the aforesaid cellulose ethers; cellulose acetate phthalate; hydroxypropyl methyl cellulose phthalate; hydroxypropyl methyl cellulose acetate succinate; ethyl cellulose; carboxymethyl hydroxyethyl cellulose and a sodium salt of a carboxymethyl hydroxyethyl cellulose.

A further aspect of the present embodiment optionally provides that said film further comprises a plasticiser. Optionally, said plasticiser comprises one or more of: polyols; glycols; acetins; carboxylic acid; and esters of carboxylic acid, and more particularly said plasticiser comprises one or more of: polyethylene glycol; glycerine; triacetin; citric acid; and triethyl citrate. Optionally, said plasticiser or plasticisers at present in an amount up to 40% of the solid film. Preferably, said plasticiser is present in an amount up to 20% of the solid film.

A further aspect of the present embodiment optionally provides that said film, prior to the application of said fluid, comprises one or more additional active materials.

A further aspect of the present embodiment optionally provides that said fluid further comprises one or more additional active materials.

A further aspect of the present embodiment optionally provides that said method further comprises the application of a one or more additional fluids comprising one or more additional active materials to the surface of said water-soluble film. Optionally, at least one of said one or more additional active materials has a different distribution on a surface of said film in comparison with the distribution of said nicotine.

Optionally, one or more additional active materials is present at locations arising from the application of a fluid comprising said one or more additional active materials, such that said film surface comprises areas of demarcation, wherein one or more of said areas comprises a measurably lower concentration of said one or more additional active materials in relation to one or more other said areas. Optionally, said one or more areas comprises a concentration of said one or more additional active materials of 0%. Optionally, said areas of demarcation comprising one or more additional active materials exactly match the locations at which nicotine is present. Alternatively, optionally, said areas of demarcation comprising one or more additional active materials overlap with the locations at which nicotine is present. Still alternatively, optionally, said areas of demarcation comprising one or more additional active materials partially overlap with the locations at which nicotine is present. Still further alternatively, optionally said areas of demarcation comprising one or more additional active materials are distinct from the locations at which nicotine is present.

Optionally, one or more of said active materials comprises caffeine. Optionally, one or more additional active materials comprises an analgesic. Optionally, one or more additional active materials comprises a breath freshening agent. Preferably, said breath freshening agent comprises menthol. Optionally said one or more additional active materials comprises an oral cleansing agent. Preferably, said oral cleansing agent comprises one or more of a quaternery or ammonium base.

A further aspect of the present embodiment optionally provides that said water-soluble film further comprising one or more of colourants, emulsifiers, humectants, defoamers and anti-block agents.

An alternative embodiment of the present invention provides a method for producing a water-soluble film comprising nicotine, including the steps of:
Preparing an aqueous polymer solution;
Casting a film from said aqueous solution;
Drying said cast film; and
Applying a fluid containing nicotine in a pharmacologically effective form to the dried film,
Wherein the step of applying is performed using jet-based spraying technology capable of application of predetermined amounts of fluid on predetermined areas of said film surface.

A further aspect of the present embodiment optionally provides that said pharmacologically active form of: nicotine is selected from one or more of: a nicotine oil; a nicotine salt; or a complex of nicotine. Optionally, said pharmacologically active form of nicotine is nicotine bitartrate dehydrate. Optionally, said nicotine salt is: a hydrochloride, dihydrochloride; sulphate, tartrate; ditartrate; zinc chloride; salicylate; alginate; ascorbate; benzoate; citrate; edetate; fumarate; lactate; maleate; oleate; or sorbate. Optionally, said pharmacologically active form of nicotine comprises a microencapsulated form of nicotine.

A further aspect of the present embodiment optionally provides that said fluid is a liquid. Optionally said liquid comprises a solution, a suspension or a micro-emulsion of said nicotine.

A further aspect of the present embodiment optionally provides that said fluid is an aqueous solution, said pharmacologically active form of nicotine is nicotine bitartrate dehydrate, and said solution further comprises hydroxyl ethyl cellulose, Pullulan, polyvinyl pyrollidone, and propylene glycol alginate.

A further aspect of the present embodiment optionally provides that said fluid is applied onto only one surface of said water-soluble film.

A further aspect of the present embodiment optionally provides that said predetermined amount of fluid provides said water-soluble film with an accurately metered dose of said nicotine. Preferably, said film is divided into individual dosage units, and wherein said fluid is applied in an amount such that each said individual dosage unit comprises a dose of said nicotine of up to 2 mg. More preferably, each said individual dosage unit comprises a dose of said nicotine of between 0.1 and 1.0 mg. More preferably still, each said individual dosage unit comprises a dose of said nicotine of 0.4-0.7 mg.

A further aspect of the present embodiment optionally provides that said fluid is applied in an even distribution on the surface of said film.

A further aspect of the present embodiment optionally provides that said fluid acts as an adhesive to enable said water-soluble film to be bonded to a substrate. Optionally, said substrate comprises one or more additional layers of film. Preferably, one or more of said additional layers of film comprise one or more of films made in accordance with any of the above embodiments, and/or films according to any of the above embodiments. Optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film exactly or substantially match the location of the nicotine at one or more layers of said substrate. Alternatively, optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film overlap with the location of the nicotine at one or more layers of said substrate. Still alternatively, optionally, one or more layers of said substrate comprise nicotine, and wherein said fluid is applied only to one or more predetermined discrete areas of the surface of said water-soluble film, such that when said film is bonded to said substrate, said discrete areas of the surface of the water-soluble film partially overlap with the location of the nicotine at one or more layers of said substrate. Still further alternatively, optionally, wherein one or more layers of said substrate comprise nicotine, and wherein said fluid is applied only to one or more predetermined discrete areas of the surface of said water-soluble film, such that when said film is bonded to said substrate, said discrete areas of the surface of the water-soluble film is wholly offset from the location of the nicotine at one or more layers of said substrate.

A further aspect of the present embodiment optionally provides that said water-soluble film is folded after the application of said solution, so as to create a multiple layer film. Optionally, said fluid acts to bind said film to itself. Optionally, said fluid is applied only to one or more predetermined discrete areas of the surface of said water-soluble film, such that when said film is folded to create a multiple layer film, said discrete area or areas of on the surface of the water-soluble film in one layer exactly or substantially match said discrete area or areas of on the surface of the water-soluble film in one layer at one or more other layers of said film. Alternatively, optionally, said fluid is applied only to one or more predetermined discrete areas of the surface of said water-soluble film, such that when said film is folded to create a multiple layer film, said discrete area or areas of on the surface of the water-soluble film in one layer overlap with said discrete area or areas of on the surface of the water-soluble film in one layer at one or more other layers of said film. Still alternatively, optionally, said fluid is applied only to one or more predetermined discrete areas of the surface of said water-soluble film, such that when said film is folded to create a multiple layer film, said discrete area or areas of on the surface of the water-soluble film in one layer partially overlap with said discrete area or areas of on the surface of the water-soluble film in one layer at one or more other layers of said film. Still further alternatively, optionally, said fluid is applied only to one or more predetermined discrete areas of the surface of said water-soluble film, such that when said film is folded to create a multiple layer film, said discrete area or areas of on the surface of the water-soluble film in one layer are wholly offset from said discrete area or areas of on the surface of the water-soluble film in one layer at one or more other layers of said film.

A further aspect of the present embodiment optionally provides that, after the application of said fluid to said water-soluble film, said film is cured. Optionally said curing occurs at ambient humidity. Optionally, said curing occurs at ambient temperature. Alternatively said curing is achieved by heating said substrate. Optionally, said substrate is heated to a temperature of 80° C.

A further aspect of the present embodiment optionally provides that said fluid comprises fugitive solvents.

A further aspect of the present embodiment optionally provides that said film is ingestible.

A further aspect of the present embodiment optionally provides that said water-soluble film comprises a cellulose ether.

A further aspect of the present embodiment optionally provides that said water-soluble film comprises a polymer based film comprising one or more of: hydroxy propylmethyl cellulose; hydroxy propyl cellulose; hydroxy ethyl methyl cellulose; hydroxyethyl cellulose; methyl cellulose; carboxymethyl cellulose; sodium carboxymethyl cellulose; a salt or derivative of any of the aforesaid cellulose ethers; cellulose acetate phthalate; hydroxypropyl methyl cellulose phthalate; hydroxypropyl methyl cellulose acetate succinate; ethyl cellulose; carboxymethyl hydroxyethyl cellulose and a sodium salt of a carboxymethyl hydroxyethyl cellulose.

A further aspect of the present embodiment optionally provides that said film further comprises a plasticiser. Optionally, said plasticiser comprises one or more of: polyols; glycols; acetins; carboxylic acid; and esters of carboxylic acid, more particularly one or more of: polyethylene glycol; glycerine; triacetin; citric acid; and triethyl citrate. Optionally, said plasticiser or plasticisers at present in an amount up to 40% of the solid film. Preferably, said plasticiser is present in an amount up to 20% of the solid film.

A further aspect of the present embodiment optionally provides that said film, prior to the application of said fluid, comprises one or more additional active materials.

A further aspect of the present embodiment optionally provides that said fluid further comprises one or more additional active materials.

A further aspect of the present embodiment optionally provides that said method further comprises the application of a one or more additional fluids comprising one or more additional active materials to the surface of said water-soluble film. Optionally, at least one of said one or more additional active materials has a different distribution on a surface of said film in comparison with the distribution of said nicotine. Optionally, said one or more additional active materials is present at locations arising from the application of a fluid comprising said one or more additional active materials, such that said film surface comprises areas of demarcation, wherein one or more of said areas comprises a measurably lower concentration of said one or more additional active materials in relation to one or more other said areas. Preferably, said one or more areas comprises a concentration of said one or more additional active materials of 0%. Optionally, said areas of demarcation comprising one or more additional active materials exactly or substantially match the locations at which nicotine is present. Alternatively, optionally, said areas of demarcation comprising one or more additional active materials overlap with the locations at which nicotine is present. Still alternatively, optionally, said areas of demarcation comprising one or more additional active materials partially overlap with the locations at which nicotine is present. Still further alternatively, optionally, said areas of demarcation comprising one or more additional active materials are wholly offset from the locations at which nicotine is present.

Optionally, one or more of said active materials comprises caffeine. Optionally, said one or more additional active materials comprises an analgesic. Optionally, said one or more additional active materials comprises a breath freshening agent. Preferably, said breath freshening agent comprises menthol. Optionally, one or more additional active materials comprises an oral cleansing agent. Preferably, said oral cleansing agent comprises one or more of a quaternery or ammonium base.

A further aspect of the present embodiment optionally provides that said water-soluble film further comprises one or more of: colourants; emulsifiers; humectants; defoamers; and anti-block agents.

An alternative embodiment of the present invention provides a method for producing a water-soluble film comprising nicotine bitartrate dehydrate, comprising the steps of:
i. dissolving nicotine bitartrate dehydrate in an aqueous solution comprising hydroxyl ethyl cellulose, Pullulan, polyvinyl pyrollidone, and propylene glycol alginate,
ii. casting said solution into a thin film.

A further aspect of the present embodiment optionally provides that said nicotine bitartrate dehydrate is provided in an predetermined amount to produce a said water-soluble film with an accurately metered dose of said nicotine. Preferably, said film is subsequently divided into individual dosage units, and wherein said nicotine bitartrate dehydrate is provided in an amount such that each said individual dosage unit comprises a dose of said nicotine of up to 2 mg. More preferably, each said individual dosage unit comprises a dose of said nicotine of between 0.1 and 1.0 mg. Still more preferably, each said individual dosage unit comprises a dose of said nicotine of 0.4-0.7 mg.

A further aspect of the present embodiment optionally provides that said method further comprises the addition of one or more additional active materials. Optionally, said addition of one or more additional active materials is made to said solution. Optionally, said addition of one or more additional active materials is made via the application of a fluid containing one or more active materials to the surface of said cast film. Optionally, said method may result in at least one of said one or more additional active materials having a different distribution throughout said film in comparison with the distribution of said nicotine. Optionally, said one or more additional active materials are present only at discrete positions upon or within said film such that said film comprises areas of distinct demarcation, wherein one or more of said areas comprise a measurably lower concentration of said one or more additional active materials in relation to one or more other said areas. Preferably, said one or more areas comprises a concentration of said one or more additional active materials of 0%. Optionally, said areas of demarcation comprising one or more additional active materials exactly or substantially match the locations at which nicotine is present. Alternatively optionally, said areas of demarcation comprising one or more additional active materials overlap with the locations at which nicotine is present. Still alternatively, optionally, said areas of demarcation comprising one or more additional active materials partially overlap with the locations at which nicotine is present. Still further alternatively, optionally, said areas of demarcation comprising one or more additional active materials are wholly offset from the locations at which nicotine is present.

Optionally, one or more of said active materials comprises caffeine. Optionally, one or more additional active materials comprises an analgesic. Optionally, one or more additional active materials comprises a breath freshening agent. Preferably, said breath freshening agent comprises menthol. Optionally, said one or more additional active materials comprises an oral cleansing agent. Preferably, said oral cleansing agent comprises one or more of a quaternery or ammonium base.

A further aspect of the present embodiment optionally provides the addition to said solution of one or more one or more of: colourants; emulsifiers; humectants; defoamers; and anti-block agents.

An alternative embodiment of the present invention provides a film produced in accordance with the methods of any of the above described embodiments.

An alternative embodiment of the present invention provides a dosage form comprising a film produced in accordance with a method of any of the above described embodiments.

A further aspect of the present embodiment optionally provides that said dosage form comprises a single layer of said film.

A further aspect of the present embodiment optionally provides that said dosage form comprises an enrobed tablet, wherein said tablet is enrobed by a film in accordance with any of the above described embodiments.

A further aspect of the present embodiment optionally provides that said dosage form comprises a pharmaceutical capsule, wherein the walls of said capsule comprise a film in accordance with any of the above described embodiments.

A further aspect of the present embodiment optionally provides that said dosage form comprises multiple layers of film, wherein one or more of said layers comprises a film in accordance with any of the above described embodiments. Optionally, said nicotine is comprised only within internal film layers. Optionally, no area of a film comprising said nicotine is exposed to any external surface of said dosage form. Optionally, said film is folded so as to produce said dosage form comprising multiple layers of said film. Optionally, the position of said nicotine within said dosage form, the degree of exposure of said nicotine on the surface of said dosage form, and the density of film layers surrounding said nicotine are pre-determined so as to provide a desired time, rate and/or position of release of said nicotine in a user of said dosage form.

A further aspect of the present embodiment optionally provides that said dosage form is intended to release the said nicotine and/or any additional active ingredients in the user's buccal cavity. An alternative aspect of the present embodiment optionally provides that said dosage form is intended to administer the said nicotine and/or any additional active ingredients topically. A further alternative aspect of the present embodiment optionally provides that said dosage form is intended to release the said nicotine and/or any additional active ingredients in the user's vaginal, cranial, abdominal, otic, uterine, nasal, sinus, rectal, oral, ophthalmic cavity.

An alternative embodiment of the present invention provides a kit comprising a plurality of dosage forms of any of claims 34-61 or 229-240, wherein each of said plurality of dosage forms comprises a predetermined dose of nicotine, and wherein said plurality of dosage forms comprises a range of dosages of nicotine.

An alternative embodiment of the present invention provides the use of a kit according to the above embodiment to wean a nicotine addict from said addition.

An alternative embodiment of the present invention provides an apparatus for the production of a film water-soluble film comprising nicotine, wherein said apparatus comprises a conveyor belt for transporting a water-soluble film, and at least one spray jet, said spray jet positioned so as to be able to spray a fluid comprising nicotine onto the surface of said water-soluble film. Optionally, said apparatus comprises a plurality of groups of spray jets, wherein said groups are arranged in series relative to the direction of movement of the conveyor belt, and wherein each group of spray jets comprises at least one spray jet.

An alternative embodiment of the present invention provides a method of operating an apparatus according to the above described embodiment, wherein the speed at which said conveyor belt transports said water-soluble film is such that the interval during which a point on the film passes from one group of spray jets to the next group of spray jets in said series is sufficient to allow the fluid applied by said one group of spray jets to dry to a degree sufficient to permit the application of said fluid by said second group of spray jets. Optionally, said interval is at least 15 seconds.

An alternative embodiment of the present invention provides an apparatus in accordance with any of the above described embodiments, further comprising the presence of one or more heaters to assist the drying of said fluid.

An alternative embodiment of the present invention provides an apparatus in accordance with any of the above described embodiments, further comprising control means for enabling successive applications of said fluid to be applied to the same area on the surface of said film.

An alternative embodiment of the present invention provides an apparatus in accordance with any of the above described embodiments, further comprising one or more additional spray jets positioned so as to be able to spray one or more fluids comprising additional active materials onto the surface of said water-soluble film.

An alternative embodiment of the present invention provides an apparatus in accordance with any of the above described embodiments, wherein said apparatus comprises a plurality of groups of spray jets for to spraying one or more fluids comprising additional active materials, wherein said groups are arranged in series relative to the direction of movement of the conveyor belt, and wherein each group of spray jets comprises at least on spray jet.

An alternative embodiment of the present invention provides a method of operating an apparatus according to the above described embodiment, wherein the speed at which said conveyor belt transports said water-soluble film is such that the interval during which a point on the film passes from one groups of spray jets to the next group of spray jets in said series is sufficient to allow the one or more fluids comprising additional active materials applied by said one group of spray jets to dry to a degree sufficient to permit the application of said fluid by said second group of spray jets. Optionally, said interval is at least 15 seconds.

An alternative embodiment of the present invention provides an apparatus in accordance with the above described embodiments, further comprising the presence of one or more heaters to assist the drying of said fluid.

An alternative embodiment of the present invention provides an apparatus in accordance with the above described embodiments, further comprising control means for enabling successive applications of said one or more fluids comprising additional active materials to be applied to the same area on the surface of said film.

### DRAWINGS

FIG. 1 shows an apparatus according to an embodiment of the present invention for the application of a fluid comprising nicotine to a water-soluble film;

FIG. 2 shows the dispersion of nicotine particles through a water-soluble film according to an embodiment of the present invention;

FIG. 3 shows an arrangement according to an embodiment of the invention in which a water-soluble film, in which a fluid comprising nicotine has been applied to parts of both sides of the film, may be folded so as to form a multi-layer film;

FIG. 4 shows how a number of base water-soluble films to which fluid has been applied may then be cured to form a multi-layer film comprising regions of nicotine according to an embodiment of the present invention;

FIG. 5 illustrates the rolling of a film to form a coiled dosage form according to any embodiment of the present invention;

FIG. 6 illustrates a shaped multi-layer dosage form according to an embodiment of the present invention, wherein nicotine is comprised only at one area of one of the layers; and

FIG. 7 illustrates a multi-layer structure according to an embodiment of the present invention comprising nicotine and in this case several additional active materials, each of which is located at a different position within the film.

FIG. 8 shows an alternative apparatus according to an embodiment of the present invention for the application of a fluid comprising nicotine to a water-soluble film.

FIG. 9 shows an alternative apparatus according to an embodiment of the present invention for the application of a fluid comprising nicotine to a water-soluble film.

FIG. 10 shows an alternative apparatus according to an embodiment of the present invention for the application of a fluid comprising nicotine to a water-soluble film.

### DETAILED DESCRIPTION OF THE INVENTION

This invention provides water-soluble films comprising nicotine. The term “nicotine”, as used in this document, means any pharmacologically effective form of nicotine or a suitable precursor, and includes without limitation the various nicotine oils, nicotine salts and complexes of nicotine. Preferred nicotine salts comprise hydrochlorides, dihydrochlorides, sulphates, tartrates, ditartrates, zinc chlorides, salicylates, alginates, ascorbates, benzoates, citrates, edetates, fumarates, lactates, maleates, oleates, and sorbates. A preferred complex is NBD. Details of this complex are provided below, with details of nicotine provided for reference purposes.

| | | Nicotine Bitartrate |
|---|---|---|
| | Nicotine | Dihydrate |
| Molecular | C₁₀H₁₄N | C₁₀H₁₄N₂(C₄H₆O₆)•2H₂O |
| Formula | | |
| Relative | 162.2 | 498.44 |
| Molecular Mass | | |
| Physical form | Colourless or | White lustrous |
| | brownish | crystalline powder |
| | volatile liquid | |
| Solubility | Soluble in water | Very soluble in water or |
| | or alcohol | alcohol |

Alternatively, nicotine may be provided in an alternative form, for example nicotine microencapsulated in yeast. The microencapsulation of nicotine in yeast is generally described in WO 00/69440, the disclosures of which are incorporated herein by reference.

Any suitable water-soluble film may be used, although where the intended use of the film is for an oral dosage form, the water-soluble film is preferably ingestible. Preferred films comprise cellulose ethers, whilst particularly preferred compounds include hydroxy propylmethyl cellulose, hydroxy propyl cellulose, hydroxy ethyl methyl cellulose, hydroxyethyl cellulose, methyl cellulose, carboxymethyl cellulose, sodium carboxymethyl cellulose, a salt or derivative of any of the aforesaid cellulose ethers, cellulose acetate phthalate, hydroxypropyl methyl cellulose phthalate, hydroxypropyl methyl cellulose acetate succinate, ethyl cellulose, carboxymethyl hydroxyethyl cellulose and a sodium salt of a carboxymethyl hydroxyethyl cellulose. When a film according to an embodiment of the present invention is to be used in a dosage form for which a controlled rate or specific site of release is desired, it is preferable to select a film which will provide the appropriate solubility characteristics to achieve the desired rate of release of nicotine. For example, where rapid release of the nicotine into the buccal cavity is desired, it is preferable to use a water-soluble film which is readily soluble, and so will dissolve quickly within the mouth.

These films may additionally comprise one or more plasticizers, with preferred plasticizers being polyols, glycols, acetins, carboxylic acids, and esters of carboxylic acids, and more preferably being polyethylene glycol, glycerine, triacetin, citric acid and triethyl citrate respectively. These plasticizers are preferably present in a total amount of up to 40% of the solid film, more preferably up to 20% of the solid film.

The base films may be made in a variety of ways, as would be evident to the man skilled in the art, for example, by dissolving or dispersing the film components in solvents before casting and drying the solution into a film form. For certain applications, for example the coating of edible products, it may be desirable to spray such a solution onto a pre-existing product, such as a tablet. Alternatively, the base films may be produced via hot-melt extrusion.

A preferred method for the formulation of a base water-soluble film comprises the formulation of a solution according to the following composition:

| | |
|---|---|
| Hydroxypropyl methyl cellulose (HPMC) | 10.0% |
| Glycerin | 1.0% |
| Triethyl Citrate | 1.0% |
| Purified Water | 88% |

The HPMC should be slowly added to the water at 80° C. whilst stirring, followed by addition of the glycerin. The solution should then be cooled to 30° C. whilst maintaining agitation, to produce a colourless viscous solution, before slowly adding the triethyl citrate with gentle mixing to produce a clear viscous solution. The solution should be allowed to stand for 24 hours to allow it to naturally de-aerate. The solution can then be cast, for example on a glass plate with a doctor blade set at 1.6 mm, before air-drying for 24 hours at 25° C. and 45% relative humidity, to produce a water-soluble film suitable for use with the methods of the present invention

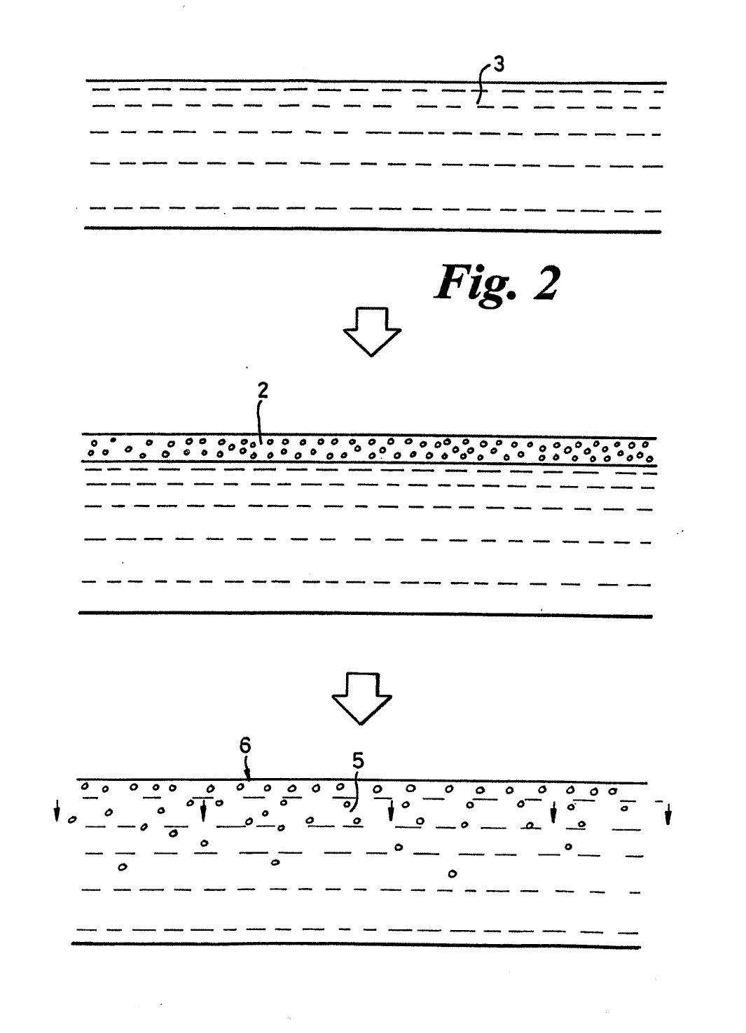

Preferably, the films of the present invention are prepared by first forming a base water-soluble film without nicotine, and subsequently applying a fluid comprising nicotine to one or more surfaces of the pre-formed water-soluble film. The surface or surfaces of the base water-soluble film are preferably dry prior to the application of the fluid. The nicotine may then reside on the surface of the film, or may be absorbed, in whole or in part into the body of the film. This can be seen in FIG. 2, wherein a fluid 2 comprising particles of nicotine 5 is applied to the surface of a base water-soluble film, 3. As shown, after the fluid 2 has been applied to the surface, the nicotine particles 5 disperse through the body of the film 3. This has the advantage that the nicotine is not exposed to the harsh conditions (for example elevated temperature) sometimes required in order to cast the base film. As such, the nicotine is less likely to be degraded.

Alternatively, films of the present invention can be made by dissolving NBD in an aqueous solution of hydroxyl ethyl cellulose, Pullulan, polyvinyl pyrollidone (“PVP”), and propylene glycol alginate (“POGA”). This solution can then be cast to form thin films of the present invention. All of the polymers in such a solution are non-ionic and do not react with the NBD. Further, it has been found that NBD does not crystallize in such a solution during formation of such a film. Films made according to the process, in addition to comprising embodiments of the present invention, can be used as the base water-soluble film to which the fluid comprising nicotine is applied. Further, or in the alternative, the aqueous solution thus described can comprise the fluid comprising nicotine for application to the base water-soluble film.

The fluid comprising the nicotine is preferably a liquid, and may comprise for example a solution, a suspension or a micro-emulsion of the nicotine. The fluid may optionally include fugitive solvents, i.e. solvents which leave the solution, for example by evaporation from the solution. Preferably, the fluid will include at least one polymer compatible with the base film.

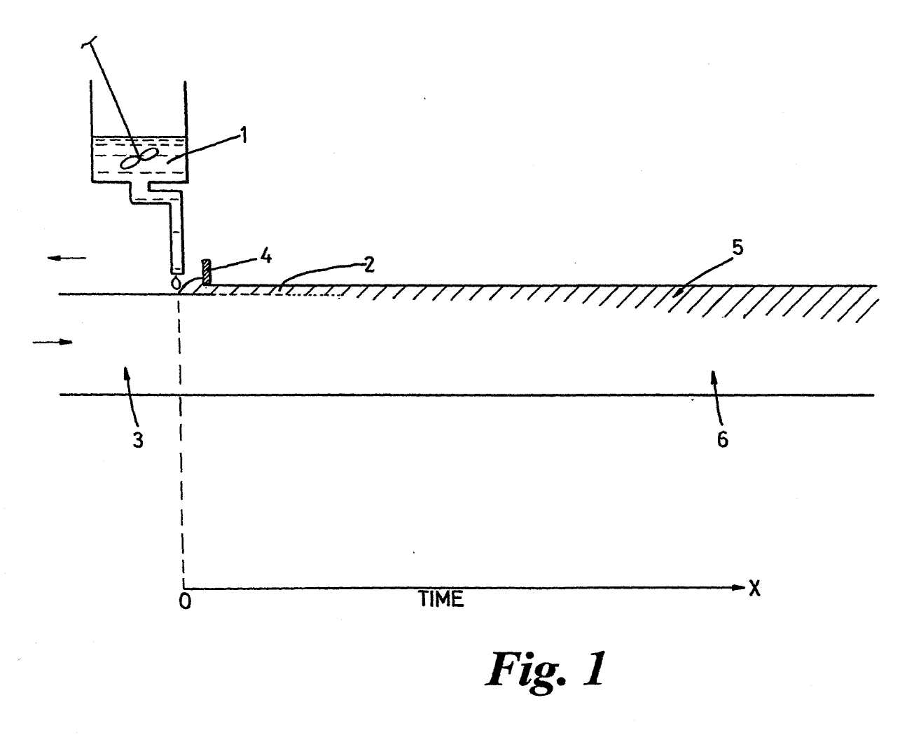

There are various ways in which the fluid may be applied to the surface of the base film, as will be readily understood by the person skilled in the art. For example, as seen in FIG. 1, application of liquid fluid 1 may be achieved by applying the fluid under the control of a doctor blade 4. Alternative methods of application can include application via extrusion, roller means, spraying, brush painting and wiping.

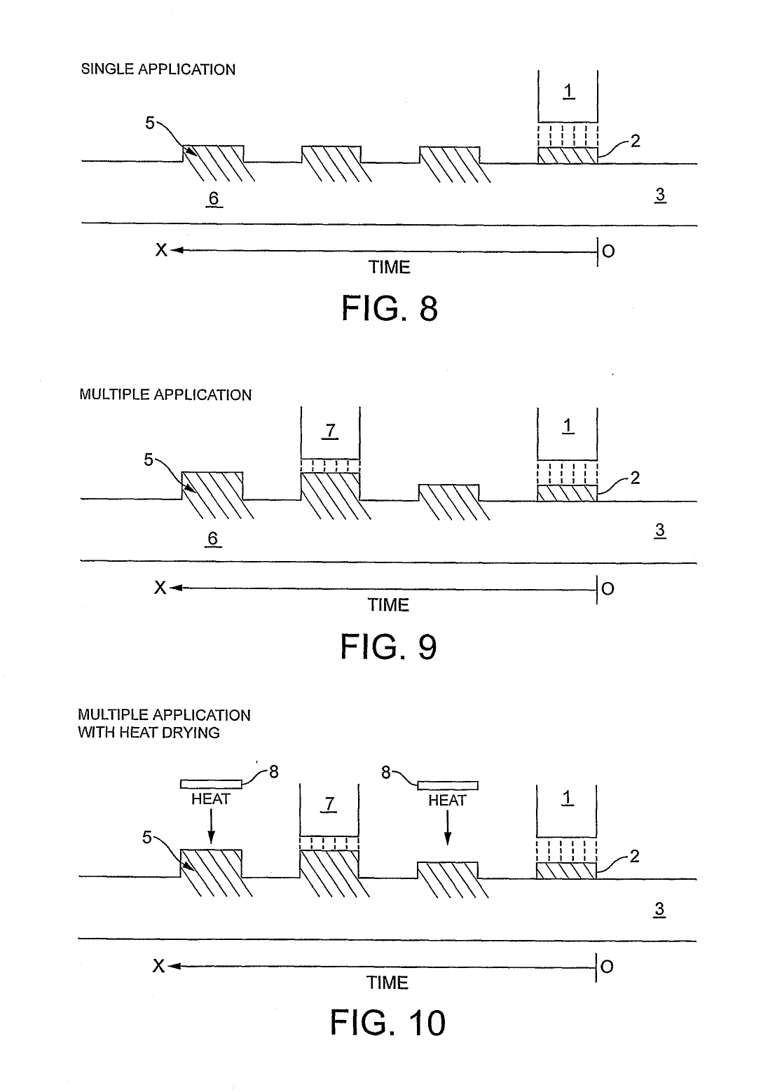

A particularly preferred method of application of the fluid is via the means of one or more spray jets, such as those used in ink jet technology. For example, the printing system provided by a standard commercial inkjet printer can be used to apply the fluid. Apparatus comprising such spray jets can be seen in FIG. 8, wherein the base water-soluble film 3 is represented as moving from right to left, under a spray jet 1 (which may be a single spray jet or a group of spray jets). Spray jet 1 applies fluid 2 comprising nicotine 5 to the surface of water-soluble film 3, and over time nicotine 5 disperses into water-soluble film 3. When applying a liquid fluid by this method, the viscosity of the liquid and the precise solvents used will need to be compatible with the inkjets, although this can be easily achieved by a man skilled in the art.

Multiple applications of the fluid may be necessary, depending upon the desired dosage of nicotine the film is intended to provide, and also on the extent to which the particular form of nicotine is soluble in the particular solvent. Based on these criteria, the man skilled in the art would be readily able to calculate the necessary number of applications, with 1-10 applications being common, and at least 2 being preferable. Where multiple applications are provided, it is preferable to allow a previous application to dry, either partially or in total, before providing a further application of fluid. An interval of at least 15 seconds between applications is preferable. Advantageously, the applications may be provided in register with each other, such that each successive application of fluid is applied to the same surface area of the base water-soluble film as that to which the previous application of fluid was applied.

When providing multiple applications of fluid, it may be desirable to provide a conveyor-based apparatus for the production of the film comprising multiple groups of spray jets. In such an apparatus, the base film is moved along a conveyor belt, passing under a plurality of groups of inkjets (each group comprising at least one inkjet). The groups of inkjets may be arranged in series relative to the direction of movement of the conveyor belt, and the speed at which the film moves along the conveyor belt may be such that it provides that the interval during which a point on the film passes from a first group of inkjets to a second group of inkjets is sufficient to allow the fluid from the previous application to dry to a degree sufficient to permit the next application of the fluid. Preferably, the interval during which a point on the base film passes from one group of inkjets to the next group of inkjets in the series is at least 15 seconds.

Such an apparatus is shown in FIG. 9, wherein the base water-soluble film 3 is represented as moving from right to left, under a group of spray jets 1 (which may comprise one or more spray jets). Spray jet 1 applies fluid 2 comprising nicotine 5 to the surface of water-soluble film 3. The film is then transported towards a second group of spray jets 7. The time interval during which the surface area of the film to which fluid 2 was applied is transported from the location at the first group of spray jets 1 to the location at the second group of spray jets 7 is sufficient to allow fluid 2 to at least partially dry. The second group of spray jets 7 then applies more fluid 2 to the same surface area of the film to which the first group of spray jets 1 applied the fluid 2. In this manner the amount of fluid applied to that specific area on the surface of water-soluble film 3 is increased. Over time nicotine 5 disperses into water-soluble film 3.

FIG. 10 shows a further embodiment of the present invention in which apparatus as per FIG. 9 is provided, the apparatus further comprising means 8 for heating the film 3 to assist the drying of fluid 2 on the surface of film 2 between successive applications of fluid 2.

The use of ink jet technology enables the fluid to be distributed evenly across the film, and can also enable the fluid to be applied to precise areas, the fluid being optionally evenly distributed within such areas, and even in particular patterns. This is advantageous where it is desirable to locate the nicotine only in certain areas of the film, for example if the film is to be one layer of a multi-layer film, it may be advantageous to provide nicotine only to parts of the film which will not be exposed on external surfaces of the multi-layer film, for example, so as to protect the nicotine and prevent its contamination or degradation. The use of ink jets to apply the fluid in precise locations on the film may also reduce the amount of wastage of nicotine, in comparison to other methods.

Upon application of the fluid, the nicotine may disperse through the film to a degree sufficient to provide complete homogeneity of the nicotine with the base film. Alternatively, there may be only partial absorption or dispersal of the nicotine. This will lead to the nicotine remaining substantially within the area of application, which may be advantageous, for example, when the film is to be used as part of a multi-layer film, in order to protect the nicotine. Fugitive solvents may be included within the fluid to accelerate the absorption of the nicotine.

Partial dispersal of the nicotine through the base film may prescribe a concentration gradient of nicotine through the base film, with greatest concentration being at the site of application of the fluid, with concentration decreasing with distance away from the surface of application. Such a concentration gradient may be advantageous in certain circumstances, for example when an embodiment of the present invention is to be used to coat another product, or form a wall of capsule, for example. The gradient can be particularly pronounced when the fluid is applied only to one surface of the film, as is shown in FIG. 2, in which fluid 2 is applied to base water-soluble film 3 on one side only. Nicotine 5 disperses through film 3, but only to a limited extent, the greatest concentration of nicotine being at the surface 6 to which the fluid was applied.

The nicotine is generally absorbed by the film to a degree sufficient to ensure that only a single layer of film exists (as opposed to a two layer film comprising a base film layer and a fluid/nicotine layer).

The fluid can be applied to the film in a predetermined amount, such that where the film is to be used as, or in the formation of dosage forms, each dosage form provides a known, predetermined dose of nicotine. Preferably, such a dosage form provides a dose of nicotine of up to 2 mg; more preferably the dose of nicotine is between 0.1 and 1 mg mg; and more preferably still the dose of nicotine is of 0.5 mg. Although the dose provided by cigarettes varies between different types of cigarette, on average, a cigarette provides a dose of nicotine of approximately 0.7 mg over the period taken to smoke a cigarette, e.g. 4 minutes. Because a suitably water-soluble film or dosage form embodying the present invention can provide a dose more quickly, the dose can be lower, such that a dose of 0.5 mg may be sufficient to mimic the dose of an average cigarette in a user. As the user becomes weaned from their addiction, it may be preferable to provide a lower dose to such a user, to assist the weaning process, such that doses between 0.5 mg and 0.1 mg may be preferred.

Alternatively, there may be users who have no intention of giving up nicotine, and instead enjoy the pharmacological effects provided by nicotine. In such cases, a user may enjoy a dosage form of the present invention comprising a dose of nicotine equivalent to or greater than that of a normal cigarette. For example certain users may enjoy a dose of nicotine of up to 2 mg or more.

After application of the fluid to the base film, the film may be cured. This may occur at an elevated temperature, for example a temperature of up to 80° C., in order to speed the curing process. Alternatively, curing may occur at ambient temperature, and/or ambient humidity, in order to protect the nicotine, and any other active materials include in the film, from degradation.

Films of the present invention can be used to form part of a multi-layer film structure. Such a multi-layer film structure can contain one or more films of the present invention. Alternatively and/or additionally, multiple layers can be formed by a single film of the present invention, by folding the film in a desired manner.

When it is intended for a film of the present invention to be included as part of a multi-layer film, the fluid may be applied to the film only in areas of the film which will be encompassed by the additional layers of film, i.e. one or more of the surfaces which represent internal rather than external film surfaces of the finished multilayer film structure.

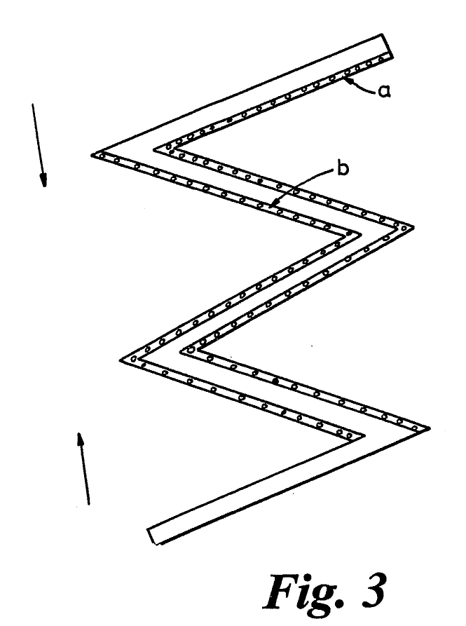

For example, as shown in FIG. 3, where folding of a single film into a multi-layer film structure is intended, the fluid may be applied only to areas on the surfaces a, b of the base water-soluble film which will be encompassed internally after the film is folded.

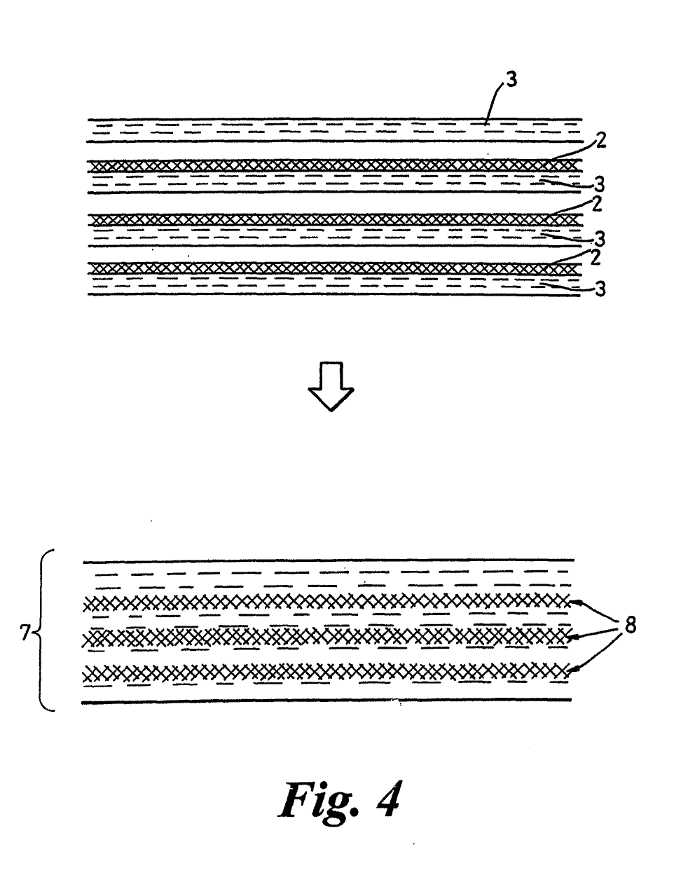

Alternatively, as shown in FIG. 4, the same may be achieved by applying the fluid 2 only to a single surface of each of a plurality of base water-soluble films 3, prior to layering said films. In such cases, the nicotine 8 will be located internally in the multilayer film, such that no nicotine is exposed on the external surfaces of the multi-layer film.

Such approaches protect the nicotine (and any other active materials thus applied) from contamination and/or degradation arising due to exposure to external environments. Additionally, by encompassing the nicotine in multiple layers of film, the rate or time of release of the nicotine may be controlled, due to the time taken for the outer layers of film to dissolve. Further, layers of film with different solubility characteristics, for example films which dissolve only in acidic conditions such as those found in the stomach, may be provided so as to control the position of the release of nicotine within the user.

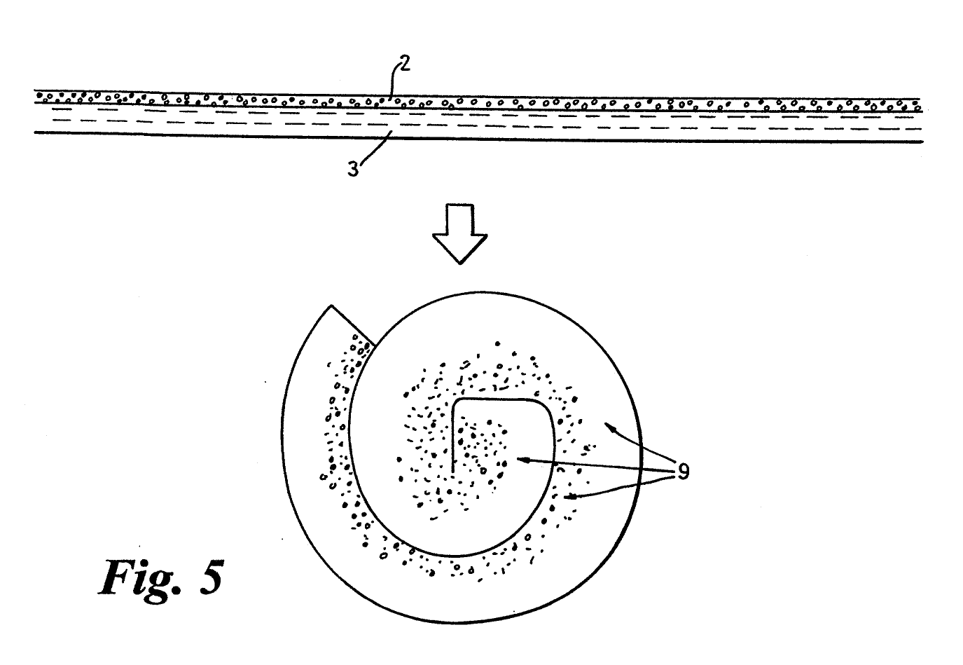

The same effect of internalising the applied nicotine may also be achieved by rolling a film of the present invention, as shown in FIG. 5, wherein a base water-soluble film 3 has fluid 2 applied only to a single surface. This film is then rolled to produce a coiled dosage form in which the nicotine 9 is located substantially internally within the dosage form.

When forming a film into a multi-layer structure according to an embodiment of the present invention, an additional adhesive may be used to bind the layers of film together. More preferably, though, the fluid applied to the film may act as an adhesive, either inherently, for example due to the presence of a polymer compatible with the base water-soluble film, or via the presence of additional adhesive components known to a skilled person, such as a dilute solution comprising similar constituents to those contained within the base films, such as propylene glycol. This is particularly advantageous where a single film is folded to create a multi-layer film structure according to an embodiment of the present invention, where the fluid acts as an adhesive, enabling the film to bind to itself.

It may be desirable according to an embodiment of the present invention to bind a film to a substrate, for example a tablet or a foodstuff. In such instances a separate adhesive may be used. Alternatively, it may be appropriate in certain instances for the fluid to act as an adhesive for binding the film to the substrate. This will depend on the nature of the substrate, fluid and film in each case, as would be readily understood by the person skilled in the art.

It may be desirable to include further active ingredients, in addition to nicotine, within the films of the present invention. Such active ingredients may comprise caffeine, analgesics, for example, ibuprofen, breath freshening agents, for example menthol, oral cleansing agents, for example a quaternary or ammonium base, or other suitable compounds conferring a desirable property upon the film.

These further active ingredients may be incorporated in the base water-soluble film prior to the application of the fluid. Alternatively, and or additionally, further active materials may be included in the fluid comprising the nicotine. In this case, the active material and the nicotine may have different dispersal properties, such that the extent of dispersal of the nicotine and the active material may vary, for example only one material may achieve full homogeneity through the base film, only one material may provide a gradient of concentration, etc. As an alternative to, or in addition to either of the previously described methods of providing further active materials, a second fluid comprising one or more further active materials may be applied to the water-soluble film, either before or after the application of the fluid comprising nicotine. This fluid may be provided via the same, or different means as the fluid comprising nicotine, and if applied by means of ink jet type technology, may be applied to precise areas or in particular patterns. Such areas or patterns may exactly match, partially or entirely overlap with or be deliberately distinct from areas of application of nicotine. Embodiments thus provide a way of controlling areas of a surface to which fluid(s) is applied in 2 dimensional space. The fluid may be provided in predetermined amounts so as to provide a precise dosage of the further active material, if necessary.

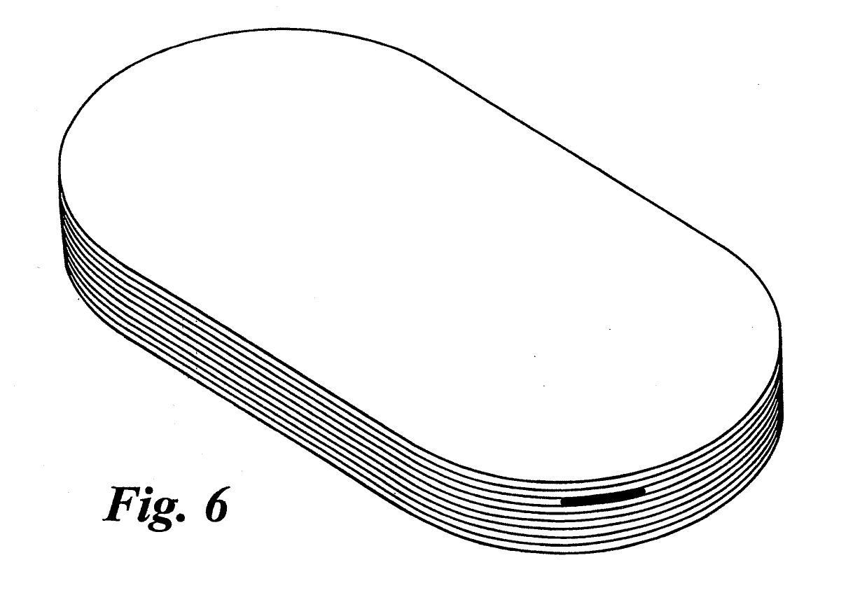

FIG. 6 illustrates a multilayer film provided with a shape beneficial for oral administration, in which part of one of the internal layers of the multilayer film comprises nicotine

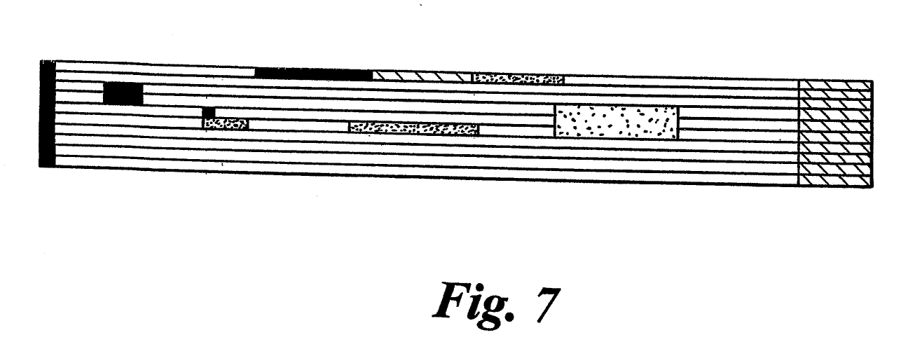

FIG. 7 illustrates an exemplary multilayer film produced in accordance with the present invention, comprising nicotine and in this case 3 other active ingredients (indicated by the differently shaded regions), each having a different predetermined location in 3 dimensional space within the multilayer film. This 3 dimensional distribution of components is achieved by to the application of the active ingredients to different areas of the surfaces of the film, and in this case, also to different film surfaces of the various layers within the multi-layer film.

The films of the present invention may further comprise colourants, flavourings, emulsifiers, humectants, defoamers and anti-block agents as desired. Such options and suitable substances are generally known to a skilled person.

An exemplary composition of a film produced in accordance with the present invention is as follows:

| | |
|---|---|
| HPMC | 30% |
| Microcrystalline cellulose | 15% |
| Starch | 10% |
| Maltodextrin | 10% |
| Gum Arabic | 10% |
| Vegetable oil | 6% |
| Sorbitol | 4% |
| Flavour | up to 15% |
| Nicotine (in a desirable form, for example NBD) | up to 5% |

Alternative compositions for films produced in accordance with the present invention, wherein the form of nicotine provided may be nicotine oil or nicotine microencapsulated in yeast, are as follows:

| | |
|---|---|
| Natrosol 250 g pharm | 39.25% |
| Avicel LM310 | 28.00% |
| Glycerin | 11.00% |
| Maltodextrin | 6.75% |
| Gum Arabic | 9.00% |
| Cinnamon Flavour | 4.75% |
| Nicotine | 1.25% |

| | |
|---|---|
| Natrosol 250 g pharm | 34.25% |
| Avicel LM310 | 24.50% |
| Glycerin | 9.80% |
| Maltodextrin | 5.85% |
| Gum Arabic | 7.85% |
| Peppermint Flavour | 16.50% |
| Nicotine | 1.25% |

| | |
|---|---|
| Natrosol 250 g pharm | 34.25% |
| Avicel LM310 | 24.50% |
| Glycerin | 9.80% |
| Maltodextrin | 5.85% |
| Gum Arabic | 7.85% |
| Spearmint Flavour | 16.50% |
| Nicotine | 1.25% |

Films according to embodiments of the present invention, including films made in accordance with any of the methods described above, may be used for a variety of purposes, but a preferable use envisaged by the present invention is the use of such films as dosage forms and/or in the production of dosage forms.

Such a dosage form may comprise a single layer of a film in accordance with the present invention. Alternatively, the dosage form may comprise a multi-layer film, which in turn comprises one or more films of the present invention. For certain multi-layer dosage forms, it may be advantageous to provide a dosage form wherein the nicotine is comprised internally within the dosage form. In particular, it may be advantageous to provide a dosage form wherein no nicotine is present on the external surface of the dosage form. For particular dosage forms, it may be desirable to produce a dosage form wherein the degree of exposure of the nicotine, and the density of film surrounding the nicotine are predetermined, so as to control the rate, time and/or position of the release of the nicotine within a user. This can be done for example by providing layers of film within the dosage form which encompass the nicotine containing layer, and which only dissolve under certain conditions, for example under conditions of high acidity as found in the stomach, or by providing encompassing layers of such density or solubility that they will take a certain, predetermined time to dissolve in the buccal cavity or other envisaged site, such that the nicotine-containing layer will not be exposed until a certain period has elapsed. Further and/or alternatively, the nicotine may be encompassed in a water-soluble layer which dissolves only at a known rate, such that the nicotine is only released from the water-soluble film into the patient at the rate of dissolution of the water-soluble film. Similar methods may be used to control the time, rate and/or position of release of any additional active materials included in the dosage form, which may be the same as or differ from the time, rate and/or position of release of the nicotine.

Such a dosage form may be a dosage form for the topical administration of nicotine, and or may be intended to release the nicotine (and/or any additional active ingredients) in any of the user's vaginal, cranial, abdominal, otic, uterine, nasal, sinus, rectal, oral or ophthalmic cavities.

Particularly preferred is a dosage form intended for release of the nicotine in the user's buccal cavity. This is especially advantageous when the dosage form is intended to provide a rapid release of the nicotine, for example by providing a thin dosage form comprising a water-soluble film which is highly soluble in the buccal cavity. This is more especially advantageous when the films provided by embodiments of the present invention are highly muco-adhesive (as are the specific films described above), such that they readily adhere to the inside of the mouth, and/or to the gums, of the user. Most advantageous is the provision of such a dosage form which provides a dose of nicotine to a user at a rate similar to or more quickly than that of a cigarette. The dose provided may also be similar to that of a cigarette. Advantageously, a series of dosage forms comprising a range of dosages of nicotine may be provided, wherein said dosage decreases in accordance with a reduction in the user's craving for nicotine, for example as the user successfully weans himself off his addition to nicotine.

In addition to films according to embodiments of the present invention comprising dosage forms, they may also comprise part of the dosage form. For example, such films may be advantageously used to enrobe tablets or other medicaments. Additionally, films according to embodiments of the present invention may be used to provide, or be used as part of, walls of a capsule, such as a liquid filled capsule, and/or a pharmaceutical capsule.

Films and dosage forms according to embodiments of the present invention may be used in the manufacture of a medicament, in particular, in the manufacture of a medicament for the treatment of nicotine addiction. Such a medicament may be intended to provide relief from the symptoms of nicotine addiction.

Films and dosage forms according to embodiments of the present invention may also be used to prevent bad breath by encouraging a user to abstain from smoking, a common cause of halitosis. The use of films according to embodiments of the present invention to prevent bad breath may further be improved by the addition of a breath cleansing agent, such as menthol, within the films according to embodiments of the present invention.

Films and dosage forms according to embodiments of the present invention may also be used to treat and for the treatment of nicotine addiction. Films and dosage forms according to embodiments of the present invention may also be used to provide relief from the symptoms of nicotine addiction.

Any of the uses of the preceding three paragraphs may comprise dissolving a film according to an embodiment of the present invention, partially or in total, within the buccal cavity of the user.

The methods, films, dosage forms, kits and uses according to embodiments of the present invention have been described in relation to nicotine. However, as would be apparent to a person skilled in the art, nicotine could be replaced by one or more alternative active ingredients in any of the methods, films, dosage forms and kits according to embodiments of the present invention, as is desirable.

1-254. (canceled)

255. A water-soluble film comprising nicotine, wherein said nicotine may be any pharmacologically effective form of nicotine present at one or more locations of the film defined by the application of a nicotine-containing fluid to at least one surface of a preformed water-soluble film.

256. The film according claim 255, wherein said nicotine is present in one or more locations arising from application of said fluid on at least one discrete area of a surface of said film such that said film surface comprises areas of demarcation, wherein one or more of said areas comprises a measurably lower concentration of said nicotine in relation to one or more other said areas.

257. The film according to claim 255 or claim 256, wherein said pharmacologically active form of nicotine is nicotine bitartrate dihydrate.

258. The film according to claim 255 or claim 256, wherein said film further comprises one or more additional active materials, and wherein said one or more additional active materials is present at locations arising from the application of a fluid comprising said one or more additional active materials, such that the film surface comprises areas of demarcation, wherein one or more of the said areas comprises a measurably lower concentration of said one or more additional active materials in relation to one or more other said areas.

259. The film according to claim 255 or claim 256, wherein said film is ingestible.

260. The film according to claim 255 or claim 256 wherein said water-soluble film comprises a cellulose ether.

261. The film according to claim 255 or claim 256, wherein said water-soluble film comprises a polymer based film comprising one or more of: hydroxy propylmethyl cellulose; hydroxy propyl cellulose; hydroxyethyl methyl cellulose; hydroxyethyl cellulose; methyl cellulose; hydroxymethyl cellulose; sodium hydroxymethyl cellulose; a salt derivative of any of the aforesaid cellulose ethers; carboxymethyl hydroxyethyl cellulose; and a sodium salt of a carboxymethyl hydroxyethyl cellulose.

262. The film according to claim 255 or claim 256, wherein said film has solubility characteristics suitable for providing a desired rate of release of said nicotine.

263. A dosage form comprising:
a water-soluble film comprising nicotine, wherein said nicotine may be any pharmacologically effective form of nicotine present at one or more locations of the film defined by the application of a nicotine-containing fluid to at least one surface of a preformed water-soluble film.

264. A dosage form according to claim 263, wherein said dosage form comprises multiple layers of film, wherein one or more of said layers comprises the water-soluble film comprising nicotine.

265. A dosage form according to claim 263 or claim 264, wherein no area of a film comprising said nicotine is exposed at an external surface of said dosage form.

266. A dosage form according to claim 263, or claim 264, wherein said film comprising said nicotine is folded so as to produce said dosage form comprising multiple layers of said film.

267. A dosage form according to claim 263 or claim 264, wherein said dosage form is intended to release the said nicotine in the user's buccal cavity.

268. A dosage form according to claim 267 wherein said films are muco-adhesive.

269. A method for producing a water-soluble film containing nicotine, wherein said nicotine may be any pharmacologically effective form of nicotine, comprising the step of applying a nicotine-containing fluid to the surface of a preformed water-soluble film, wherein said film is divided into individual dosage units, and wherein said fluid is applied in an amount such that each said individual dosage unit comprises a dose of said nicotine of up to 2 mg.

270. A method for producing a water-soluble film containing nicotine, wherein said nicotine may be any pharmacologically effective form of nicotine, comprising the step of applying a nicotine-containing fluid to the surface of a preformed water-soluble film, wherein said film is divided into individual dosage units, and wherein said fluid is applied in an amount such that each said individual dosage unit comprises a dose of said nicotine of more than 2 mg.

271. A method for producing a water-soluble film containing nicotine, wherein said nicotine may be any pharmacologically effective form of nicotine, comprising the step of applying a nicotine-containing fluid to the surface of a preformed water-soluble film, wherein said fluid is applied via means of one or more spray jets.

272. A method according to claim 271, wherein said fluid acts as an adhesive to enable said water-soluble film to be bonded to a substrate.

273. An apparatus for the production of a film water-soluble film comprising nicotine, wherein said apparatus comprises a conveyor belt for transporting a water-soluble film, and at least one spray jet, said spray jet positioned so as to be able to spray a fluid comprising nicotine onto the surface of said water-soluble film.

274. The film according to claim 255, wherein said water-soluble film comprises a plurality of cellulose ethers.

275. The film according to claim 255, wherein said water-soluble film comprises a cellulose ether and at least one water-soluble film-forming material.

276. The dosage form according to claim 263, wherein said water-soluble film comprises a plurality of cellulose ethers.

277. The dosage form according to claim 263, wherein said water-soluble film comprises a cellulose ether and at least one water-soluble film-forming material.

278. The method according to claim 269, wherein said water-soluble film comprises a plurality of cellulose ethers.

279. The method according to claim 269, wherein said water-soluble film comprises a cellulose ether and at least one water-soluble film-forming material.

280. The method according to claim 270, wherein said water-soluble film comprises a plurality of cellulose ethers.

281. The method according to claim 270, wherein said water-soluble film comprises a cellulose ether and at least one water-soluble film-forming material.

282. The method according to claim 271, wherein said water-soluble film comprises a plurality of cellulose ethers.

283. The method according to claim 271, wherein said water-soluble film comprises a cellulose ether and at least one water-soluble film-forming material.
